(19) United States
(12) Patent Application Publication
(10) Pub. No.: US 2018/0024621 A1
(43) Pub. Date: Jan. 25, 2018

(54) BIOLOGICAL INFORMATION PROCESSING SYSTEM, SERVER SYSTEM, AND INFORMATION PROCESSING METHOD

(71) Applicant: SEIKO EPSON CORPORATION, Tokyo (JP)

(72) Inventors: Michihiro NAGAISHI, Suwa-shi (JP); Shinji HATTORI, Ina-shi (JP)

(73) Assignee: SEIKO EPSON CORPORATION, Tokyo (JP)

(21) Appl. No.: 15/547,885

(22) PCT Filed: Jan. 21, 2016
(86) PCT No.: PCT/JP2016/000313

§ 371 Date: Aug. 1, 2017

(30) Foreign Application Priority Data
Feb. 24, 2015 (JP) 2015-034290

(51) Int. Cl. G06F 3/01; A61B 8/02; A61B 5/024; A61B 5/16; G06F 19/00; G06F 3/147

(52) U.S. Cl. CPC G06F 3/011; G06F 19/3406; G06F 3/147; A61B 5/02416; A61B 5/165; A61B 8/02

(57) ABSTRACT

A biological information processing system, a server system, an information processing method, and the like, which are capable of correctly imparting the intention of a transmitter of information to a receiver of the information without hurting the receiver's feeling, are provided.

A biological information processing system 100 includes a mental and physical information specification unit 110 that specifies mental and physical information on the basis of biological information measured by a sensing unit 310, a transmission and reception unit 130 that performs transmission and reception of information, and an information conversion unit 150 that performs a converting process of a presentation mode of received information on the basis of contents of the received information received by the transmission and reception unit 130, mental and physical information of a transmitter, and mental and physical information of a receiver.

### TECHNICAL FIELD

The present invention relates to a biological information processing system, a server system, an information processing method, and the like.

### BACKGROUND ART

Since the impartation of information involves a transmitter's intention or feeling, the transmitter's intention or feeling affects the giving and receiving of the information. Even when the information is received, a method of receiving the information is changed due to a receiver's feeling and the like, and thus it is difficult to correctly impart the transmitter's intention to the receiver.

Since human beings can perceive each other from each other's facial expression, gestures, tone of words, and the like in face-to-face communication, it is possible to exchange information in consideration of each other's intentions. On the other hand, in only the exchange of information based on a computer, it is difficult to perceive each other as in face-to-face communication of human beings, and thus the exchange of information taking each other's intentions into consideration can be hardly performed.

In order to realize the exchange of information taking each other's intentions into consideration, the following attempt has been proposed. For example, PTL 1 discloses a method of converting received data in conformity to a receiver's conditions. In addition, PTL 2 discloses a method of performing an incoming signal responding process of selecting a signal incoming sound in accordance with a transmitter's body condition such as illness.

### CITATION LIST

### Patent Literature

PTL 1: JP-A-2001-344352

PTL 2: JP-A-2004-274519

### SUMMARY OF INVENTION

### Technical Problem

As disclosed in PTL 1 and PTL 2, it is possible to consider the intention of the receiver or the transmitter by changing information received in accordance with conditions of either the receiver or the transmitter. However, in the inventions disclosed in PTL 1 and PTL 2, it is not possible to change a method of imparting information in accordance with conditions of both the transmitter and the receiver. For this reason, it is difficult to adapt to information sharing on the Internet, the release of information to many unspecified people, and the like.

### Solution to Problem

In addition, in the invention disclosed in PTL 2, even when the receiver can consider the transmitter's intention, the receiver may feel unpleasant. In contrast, in the invention disclosed in PTL 1, even when the receiver can receive information in a comfortable state, the transmitter's intention may not be correctly imparted. That is, in the inventions disclosed in PTL 1 and PTL 2, it is difficult for the receiver to consider the transmitter's intention in a comfortable state.

According to some aspects of the invention, it is possible to provide a biological information processing system, a server system, an information processing method, and the like which are capable of correctly imparting the intention of a transmitter of information to a receiver of the information without hurting the receiver's feeling.

An aspect of the invention relates to a biological information processing system including a mental and physical information specification unit that specifies mental and physical information on the basis of biological information measured by a sensing unit, a transmission and reception unit that performs transmission and reception of information, and an information conversion unit that performs a converting process of a presentation mode of received information, on the basis of contents of the received information received by the transmission and reception unit, mental and physical information of a transmitter, and mental and physical information of a receiver.

In the aspect of the invention, the converting process of the presentation mode of the received information is performed on the basis of the contents of the received information, the mental and physical information of the transmitter, and the mental and physical information of the receiver. Accordingly, it is possible to correctly impart the intention of a transmitter of information to a receiver of the information without hurting the receiver's feeling.

In addition, in the aspect of the invention, the information conversion unit may perform conversion of the presentation mode of the received information on the basis of the contents of the received information, the mental and physical information of the transmitter, the mental and physical information of the receiver, and at least one of positional information of the transmitter and positional information of the receiver.

Thereby, it is possible to perform the presentation of the received information in a presentation mode suitable for at least one of the position of the transmitter and the position of the receiver, and the like.

In addition, in the aspect of the invention, the information conversion unit may perform conversion of the presentation mode of the received information so that the received information is displayed on a display unit in at least one display mode of emphasis display, exaggeration display, and omission display.

Thereby, it is possible to perform the presentation of the received information in a display mode suitable for the contents of the information, the mental and physical information of the transmitter, and the mental and physical information of the receiver, and the like.

In addition, in the aspect of the invention, the information conversion unit may determine which one of a positive state, a normal state, and a negative state a mental state is, on the basis of at least one of the mental and physical information of the receiver and the mental and physical information of the transmitter, and may perform conversion of the presentation mode of the received information on the basis of the determined mental state.

Thereby, it is possible to perform the presentation of the received information in a presentation mode suitable for the respective mental states of the positive state, the normal state, and the negative state, and the like.

In addition, in the aspect of the invention, the information conversion unit may perform conversion of the presentation mode of the received information so that the received information is displayed on the display unit in at least one display mode of the emphasis display and the exaggeration display in a case where it is determined that the mental state is the positive state, and may perform conversion of the presentation mode of the received information so that the received information is displayed on the display unit in the display mode of the omission display in a case where it is determined that the mental state is the negative state.

Thereby, it is possible to perform the presentation of the received information by reflecting the transmitter's intention more strongly in a case where the mental state is a positive state, to perform the presentation of the received information so as not to hurt the receiver's feeling in a case where the mental state is a negative state, and the like.

In addition, in the aspect of the invention, the information conversion unit may determine which one of state of a formal state, a normal state, and a personal state a surrounding environment of the receiver is, on the basis of the positional information, and may perform conversion of the presentation mode of the received information on the basis of the determined surrounding environment.

Thereby, it is possible to perform the presentation of the received information in a presentation mode suitable for the surrounding environment of each of the formal state, the normal state, and the personal state, and the like.

In addition, in the aspect of the invention, the information conversion unit may perform conversion of the presentation mode of the received information so that the received information is displayed on a display unit in at least one display mode of emphasis display and exaggeration display in a case where it is determined that the surrounding environment is in the normal state, and may perform conversion of the presentation mode of the received information so that the received information is displayed on the display unit in the display mode of the omission display in a case where it is determined that the surrounding environment is in the personal state.

Thereby, it is possible to perform the presentation of the received information by reflecting the transmitter's intention more strongly in a case where the surrounding environment is in the normal state, to perform the presentation of the received information so as not to hurt the receiver's feeling in a case where the surrounding environment is in the personal state, and the like.

In addition, in the aspect of the invention, the information conversion unit may determine which one of formal contents, normal contents, and personal contents the contents of the received information are, and may perform conversion of the presentation mode of the received information on the basis of the determined contents.

Thereby, it is possible to perform the presentation of the received information in a presentation mode suitable for each of the formal contents, the normal contents, and the personal contents, and the like.

In addition, in the aspect of the invention, the information conversion unit may perform conversion of the presentation mode of the received information so that the received information is displayed on the display unit in at least one display mode of the emphasis display and the exaggeration display in a case where it is determined that the contents of the received information are the normal contents, and may perform conversion of the presentation mode of the received information so that the received information is displayed on the display unit in the display mode of the omission display in a case where it is determined that the contents of the received information are the personal contents.

Thereby, it is possible to perform the presentation of the received information by reflecting the transmitter's intention more strongly in a case where the contents of the received information are normal contents, to perform the presentation of the received information so as not to hurt the receiver's feeling in a case where the contents of the received information are personal contents, and the like.

In addition, in the aspect of the invention, the information conversion unit may perform conversion into the presentation mode in which at least one of a sound, light, and vibration is generated when the received information is displayed on the display unit.

Thereby, it is possible to strongly impress the receiver with the received information, rather than presenting received information by using only the sense of vision, and the like.

In addition, in the aspect of the invention, the transmission and reception unit may receive the mental and physical information of the transmitter, and the information conversion unit may perform the converting process of the presentation mode of the received information on the basis of the contents of the received information, the mental and physical information of the transmitter, and the mental and physical information of the receiver which are received by the transmission and reception unit.

Thereby, the information terminal on a transmission side and the biological information processing system on a reception side respectively specify the mental and physical information of the transmitter and the mental and physical information of the receiver, and thus it is possible to perform the dispersion of a processing load of a mental and physical information specification process, and the like.

In addition, in the aspect of the invention, the transmission and reception unit may receive the biological information of the transmitter, the mental and physical information specification unit may specify the mental and physical information of the transmitter on the basis of the biological information of the transmitter and specifies the mental and physical information of the receiver on the basis of the biological information of the receiver, and the information conversion unit may perform the converting process of the presentation mode of the received information on the basis of the contents of the received information, the mental and physical information of the transmitter, and the mental and physical information of the receiver which are received by the transmission and reception unit.

Thereby, it is possible to perform the converting process of the presentation mode of the received information, and the like even when the information terminal on the transmission side cannot specify the mental and physical information of the transmitter.

In addition, in the aspect of the invention, the information conversion unit may determine the presentation mode of the received information on the basis of a function F (S_{S}, P_{S}, S_{R}, P_{R}) using a parameter S_{S} obtained by digitizing the transmitter's mental state, a parameter P_{S} obtained by digitizing the transmitter's surrounding environment, a parameter S_{R} obtained by digitizing the receiver's mental state, and a parameter P_{R} obtained by digitizing the receiver's surrounding environment as arguments.

Thereby, it is possible to perform the conversion of the presentation mode by complexly considering the mental states and the surrounding environments of the transmitter and the receiver, and the like.

In addition, in the aspect of the invention, the function F may be a function including a term obtained by linear combination of the parameter S_{S}, the parameter P_{S}, the parameter S_{R}, and the parameter P_{R}.

Thereby, it is possible to perform the determination of the presentation mode in consideration of the transmitter's mental state, the transmitter's surrounding environment, the receiver's mental state, and the receiver's surrounding environment, and the like.

In addition, another aspect of the invention relates to a server system including a transmission and reception unit that performs communication between a first information terminal and a second information terminal, and a processing unit, in which the transmission and reception unit receives biological information of a transmitter which is measured by a sensing unit of the first information terminal and transmitted information from the first information terminal, and receives biological information of a receiver measured by a sensing unit of the second information terminal from the second information terminal, and the processing unit specifies mental and physical information of the transmitter on the basis of the acquired biological information of the transmitter, specifies mental and physical information of the receiver on the basis of the acquired biological information of the receiver, and performs a converting process of a presentation mode of the transmitted information on the basis of contents of the transmitted information, the mental and physical information of the transmitter, and the mental and physical information of the receiver.

Thereby, it is possible to perform the conversion of the presentation mode of the transmitted information while reducing processing loads of the information terminals on the transmission and reception sides, and the like.

In addition, still another aspect of the invention relates to an information processing method (a method of operating a biological information processing system) including specifying mental and physical information on the basis of biological information measured by a sensing unit, performing transmission and reception of information, and performing a converting process of a presentation mode of received information on the basis of contents of the received information, mental and physical information of a transmitter, and mental and physical information of a receiver.

### BRIEF DESCRIPTION OF DRAWINGS

FIG. 1 illustrates a system configuration example according to a first embodiment.

FIG. 2 is a diagram illustrating biological information and mental state information.

FIG. 3 is a flow chart illustrating a flow of processing in the first embodiment.

FIG. 4(A) to FIG. 4(G) are diagrams illustrating a display mode of received information.

FIG. 5 is a diagram illustrating parameters respectively indicating contents of a mental state, a position, and information.

FIG. 6 illustrates a system configuration example according to a modification example of the first embodiment.

FIG. 7 is a flow chart illustrating a flow of processing according to the modification example of the first embodiment.

FIG. 8 illustrates a system configuration example according to a second embodiment.

FIG. 9 is a flow chart illustrating a flow of processing according to the second embodiment.

FIG. 10 is a diagram illustrating a specific example of quantified mental state information.

FIG. 11 is a diagram illustrating influence on the intensity of a motion and motivation.

FIG. 12(A) and FIG. 12(B) illustrate examples of a specific configuration of a biological information detection device.

FIG. 13 illustrates an example of a specific configuration of the biological information detection device.

### DESCRIPTION OF EMBODIMENTS

Hereinafter, this embodiment will be described. Meanwhile, this embodiment described below is not unduly limited to the contents of the invention described in the appended claims. In addition, all configurations described in this embodiment are not limited to being essential constituent conditions of the invention.

### 1. Outline

In the exchange of text information, such as mail, through a computer, it is difficult for a transmitter to consider a receiver's conditions at the time of the reception of the text information, and it is also difficult for the receiver to correctly consider the transmitter's intention. This is because the transmitter and the receiver cannot perceive each other from each other's facial expression, gestures, tone of words, and the like as in face-to-face communication of human beings.

On the other hand, PTL 1 and PTL 2 described above disclose the invention in which a presentation mode of received information is changed in accordance with conditions of either the receiver or the transmitter. However, in the inventions disclosed in PTL 1 and PTL 2, it is not possible to change a method of transmitting information in accordance with conditions of both the transmitter and the receiver.

For example, in the invention disclosed in PTL 1, even when the receiver can receive information in a comfortable state, the transmitter's intention may not be correctly transmitted. Specifically, even when the transmitter intentionally transmits urgent information or information for inciting a sense of crisis to the receiver, the information not conforming to the receiver's feeling may result in a reduction (suppression) in the urgency or sense of crisis intended by the transmitter and is presented to the receiver. In this case, it is not possible to correctly impart the transmitter's intention to the receiver.

On the other hand, in the invention disclosed in PTL 2, even when the receiver can consider the transmitter's intention, the receiver may feel unpleasant. For example, in a case where the transmitter desires to make highly urgent contact, it is considered that the transmitter gives notice of the incoming of a signal incoming sound having a size based on the degree of urgency. A trouble is caused in the surroundings during a meeting of the receiver or the like, which may make the receiver feel awkward.

Consequently, in this embodiment, a conversion process is performed in a presentation (exhibition) mode of received information on the basis of contents of the received information, mental and physical information of a transmitter, and mental and physical information of a receiver. Thereby, it is possible to correctly impart the intention of the transmitter of information to the receiver without hurting the feeling of the receiver of the information.

### 2. First Embodiment

### 2.1. System Configuration Example

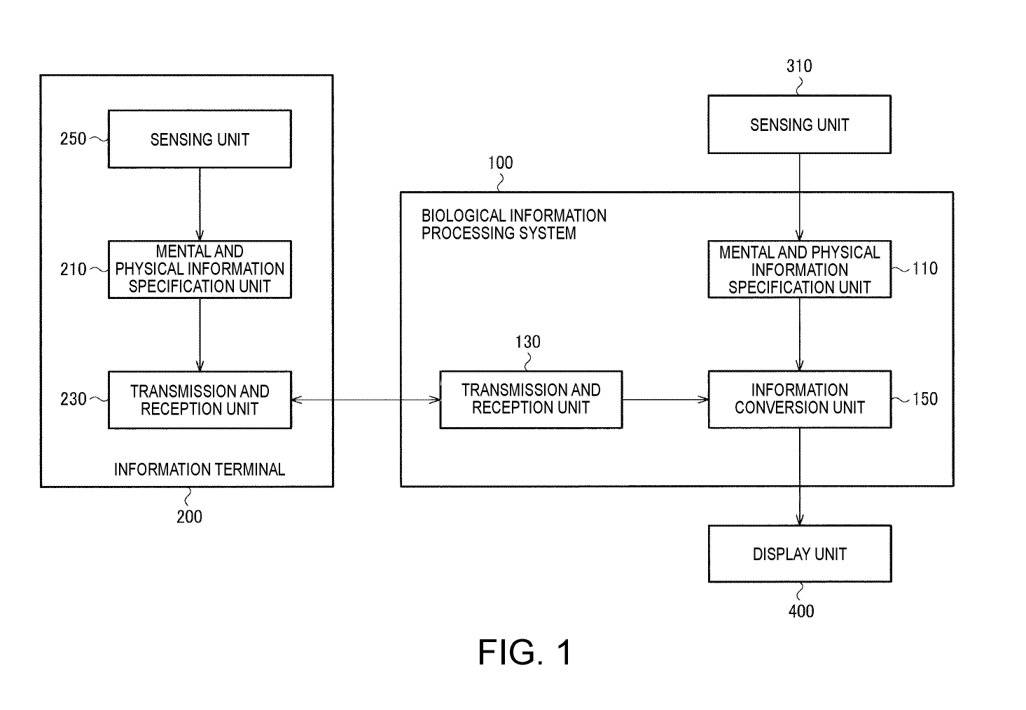

FIG. 1 is a system configuration diagram of a biological information processing system 100 and an information terminal 200 according to this embodiment. The biological information processing system 100 of this embodiment includes a mental and physical information specification unit 110, a transmission and reception unit 130, and an information conversion unit 150. In addition, the information terminal 200 is communicatively connected to the biological information processing system 100, and includes a mental and physical information specification unit 210, a transmission and reception unit 230, and a sensing unit 250. In addition, the biological information processing system 100 is connected to a sensing unit 310 and a display unit 400. Meanwhile, the biological information processing system 100 and the information terminal 200 are not limited to having a configuration illustrated in FIG. 1, and various modifications such as the omission of a portion of the components and the addition of other components can be made.

Next, processing performed in each unit will be described. Meanwhile, the mental and physical information specification unit 110, the mental and physical information specification unit 210, the transmission and reception unit 130, the transmission and reception unit 230, the sensing unit 310, and the sensing unit 250 have the same functions.

First, the mental and physical information specification unit 110 (mental and physical information specification unit 210) specifies mental and physical information on the basis of biological information measured by the sensing unit 310 (sensing unit 250). The mental and physical information specification unit 110 (mental and physical information specification unit 210) may be a communication unit (an I/F unit, an antenna unit, or the like) which transmits and receives data in a case of being connected to the sensing unit 310 (sensing unit 250) in a wireless or wired manner.

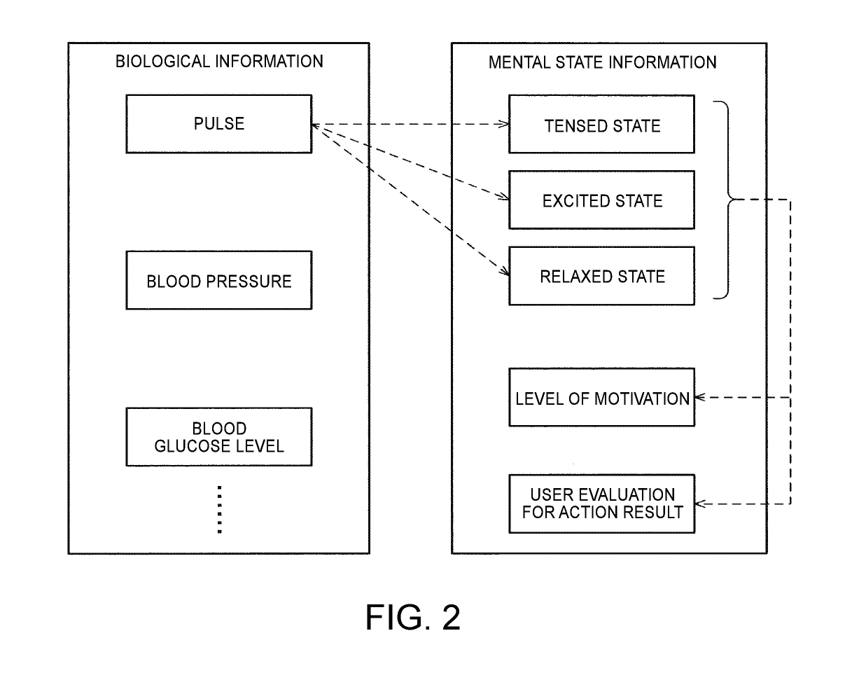

Here, the biological information includes a pulse, blood pressure, a blood glucose level, and the like as illustrated in FIG. 2. The mental and physical information is information including biological information and mental state information. The mental state information is user evaluation for a tensed state, an excited state, a relaxed state, the level of motivation, action results, and the like. However, in this embodiment, the biological information and the mental state information are not limited thereto. Meanwhile, a process of estimating mental state information will be described later in more detail.

The sensing unit 310 (sensing unit 250) is, for example, a pulse wave sensor. The pulse wave sensor is a sensor for detecting a pulse wave sensor signal, and the pulse wave sensor to be considered is, for example, a photoelectric sensor and the like. Meanwhile, in a case where the photoelectric sensor is used as the pulse wave sensor, a sensor configured to cut signal components of external light such as sunlight may be used. This can be realized, for example, by a configuration in which a plurality of photodiodes are provided and differential information is obtained through feedback processing using signals of the photodiodes, or the like.

Meanwhile, the pulse wave sensor is not limited to the photoelectric sensor, and may be a sensor using ultrasonic waves. In this case, the pulse wave sensor includes two piezoelectric elements. Here, one of the piezoelectric elements is excited to transmit ultrasonic waves to the inside of a living body, and the ultrasonic waves reflected by the blood flow of the living body are received by the other piezoelectric element. A change in frequency occurs in the transmitted ultrasonic waves and the received ultrasonic waves due to a Doppler effect of the blood flow. Also in this case, it is possible to acquire a signal corresponding to the volume of blood flow and to estimate pulsation information. In addition, another sensor may be used as the pulse wave sensor.

Further, the sensing unit 310 (sensing unit 250) includes a motion sensor, and may be able to acquire a user's body motion information. The motion sensor is, for example, an acceleration sensor. In this case, an acceleration sensor signal is detected as the body motion information. The acceleration sensor is constituted by, for example, an element of which the resistance value is increased or decreased due to an external force, and detects acceleration information of three axes. In addition, the acceleration sensor may be a six-axis acceleration sensor that detects moment around three axes in addition to a force of three axes. Further, the motion sensor is not limited to the acceleration sensor, and may be an orientation sensor such as a geomagnetic sensor, a gyro sensor, or the like. The orientation sensor measures the orientation of a sensor by an angle (0 degrees to 360 degrees). The geomagnetic sensor is constituted by, for example, an element of which the resistance value or the impedance value is increased or decreased depending on the intensity of a magnetic field, and detects geomagnetic information of three axes. In addition, a sensor having all functions of a geomagnetic sensor, an acceleration sensor, and a gyro sensor may be used.

The transmission and reception unit 130 (transmission and reception unit 230) transmits and receives information. For example, the transmission and reception unit 130 receives mental and physical information of a transmitter. In a case where the transmission and reception unit 130 and the transmission and reception unit 230 are connected to each other through a network including at least one of a wireless and wired networks, the transmission and reception unit 130 (transmission and reception unit 230) may be a communication unit (an I/F unit, an antenna unit, or the like) which transmits and receives data. Meanwhile, an example to be described below is an example in which the transmission and reception unit 230 transmits information and the transmission and reception unit 130 receives information, but the transmission and reception unit 130 may transmit information and the transmission and reception unit 230 may receive information.

In addition, the information transmitted and received by the transmission and reception unit 130 (transmission and reception unit 230) is mainly, for example, sentence (text) data, and is information which is transmittable and receivable by mail or the like. In addition, the information transmitted and received by the transmission and reception unit 130 (transmission and reception unit 230) may be an image converted into digital data, or may be sound data such as a sound. This information is called as received information when being handled by the transmission and reception unit 130, and is called transmitted information when being handled by the transmission and reception unit 230.

The information conversion unit 150 performs a conversion process in a presentation (exhibition) mode of received information received by the transmission and reception unit 130 on the basis of contents of the received information, mental and physical information of a transmitter, and mental and physical information of a receiver. Meanwhile, the function of the information conversion unit 150 can be realized by hardware such as various processors (CPU, GPU, and the like), ASIC (a gate array or the like), a program, or the like.

As described above, the information terminal 200 on a transmission side and the biological information processing system 100 on a reception side respectively specify the mental and physical information of the transmitter and the mental and physical information of the receiver, and thus it is possible to perform the dispersion of a processing load of a mental and physical information specification process, and the like.

### 2.2. Details of Processing

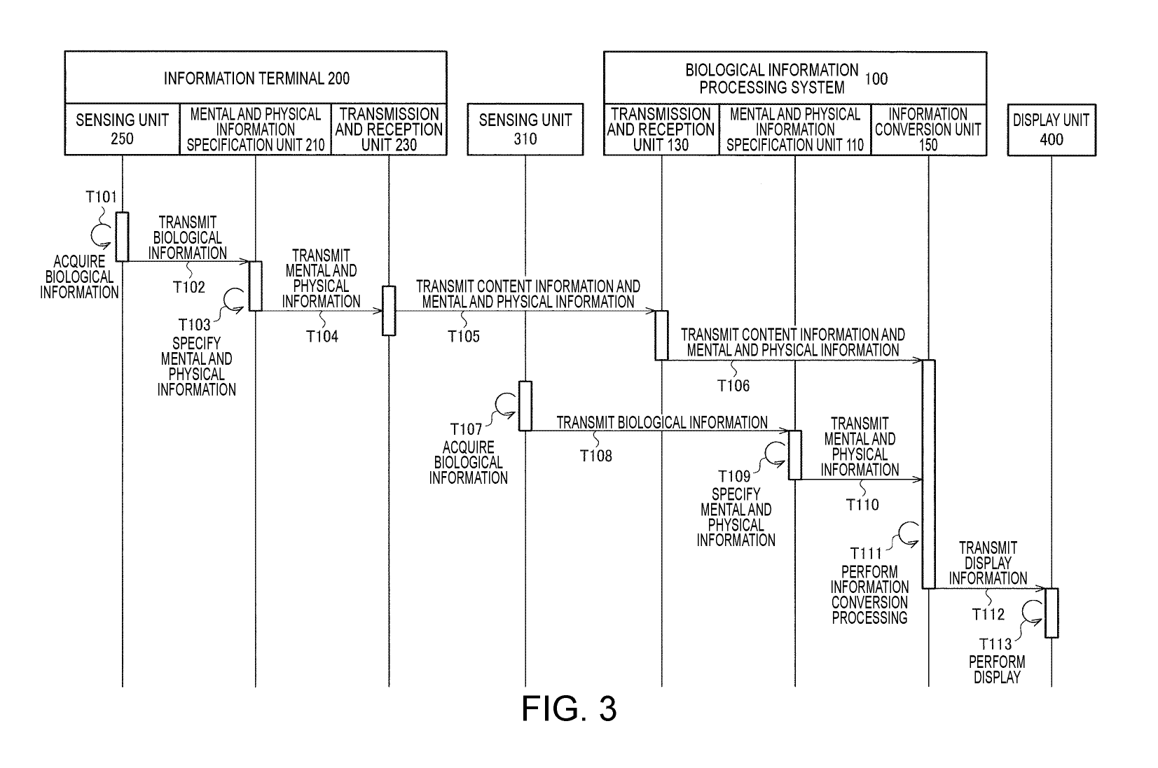

Next, a flow of processing according to this embodiment will be described in detail with reference to a sequence diagram of FIG. 3.

First, the sensing unit 250 of the information terminal 200 acquires biological information of a transmitter of information (T101), and transmits the acquired biological information to the mental and physical information specification unit 210 (T102). The mental and physical information specification unit 210 of the information terminal 200 specifies mental and physical information of the transmitter on the basis of the biological information (T103), and transmits the specified mental and physical information to the transmission and reception unit 230 (T104).

Next, the transmission and reception unit 230 of the information terminal 200 transmits content information to the transmission and reception unit 130 of the biological information processing system 100 together with the specified mental and physical information of the transmitter (T105). The content information is information such as a message (text data) or an image which is desired to be presented to the receiver by the transmitter. The content information is input to the information terminal 200 by an information acquisition unit or an input unit not shown in the drawing. The transmission and reception unit 130 of the biological information processing system 100 which has received the content information and the mental and physical information transmits these pieces of information to the information conversion unit 150 (T106).

Thereafter, the sensing unit 310 of the biological information processing system 100 acquires biological information of a receiver of information (T107), and transmits the acquired biological information to the mental and physical information specification unit 110 (T108). The mental and physical information specification unit 110 of the biological information processing system 100 specifies mental and physical information of the receiver on the basis of the biological information of the receiver (T109), and transmits the specified mental and physical information to the information conversion unit 150 (T110).

Next, the information conversion unit 150 performs conversion of a presentation mode of received information on the basis of contents of the received information (content information), mental and physical information of a transmitter, and mental and physical information of a receiver (T111), and transmits the received information of which the presentation mode has been converted to the display unit 400 (T112). At this time, the information conversion unit 150 may perform conversion of the presentation mode of the received information on the basis of at least one of positional information of the transmitter and positional information of the receiver, in addition to the above-mentioned information.

The display unit 400 displays the received information in the converted presentation mode (T113), and terminates the processing.

In the presentation mode conversion process, the information conversion unit 150 performs conversion of the presentation mode of the received information so that the received information is displayed on the display unit 400 in at least one display mode of emphasis display, exaggeration display, and omission display. The display mode is one of presentation modes.

Thereby, it is possible to perform the presentation of received information in a display mode suitable for contents of the information, the mental and physical information of the transmitter, and the mental and physical information of the receiver, and the like.

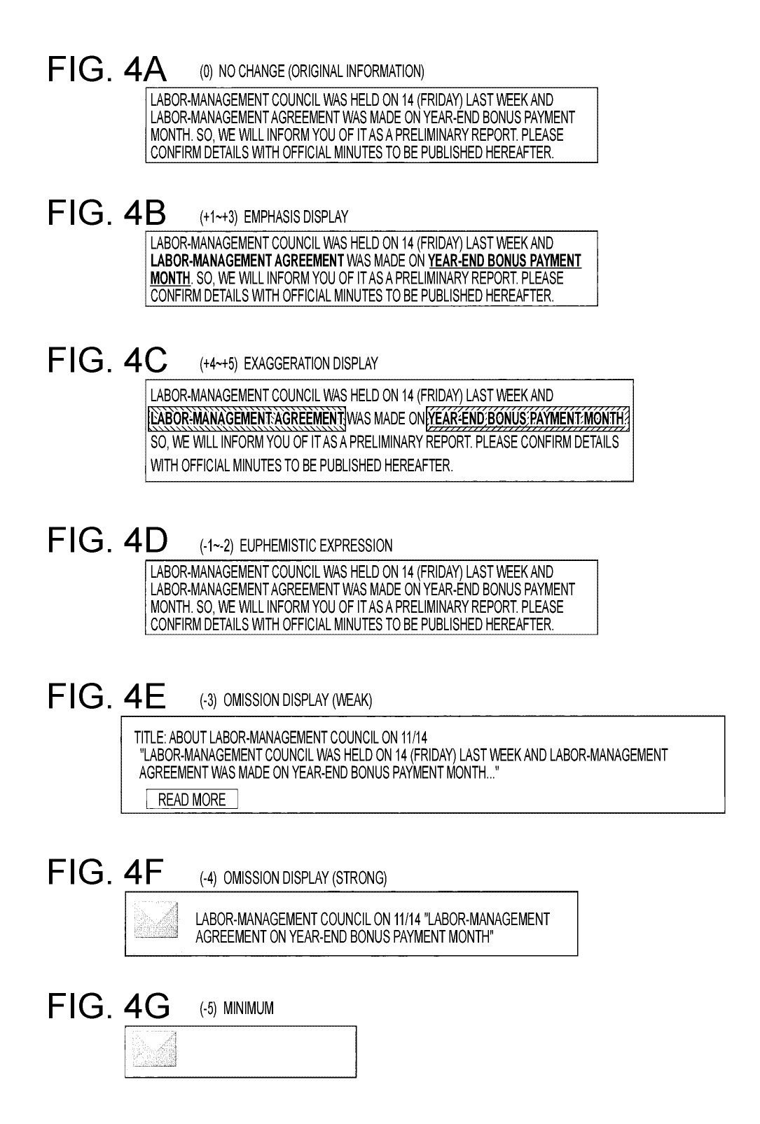

A specific example thereof is illustrated in FIG. 4(A) to FIG. 4(F). FIG. 4(A) illustrates an example in which content information (original information) transmitted by a transmitter is displayed on the display unit 400 as it is. FIG. 4(B) illustrates an example in which the original information is displayed in an emphasis display mode. In the example illustrated in FIG. 4(B), a character modification is performed by converting characters into boldface characters or enlarging a font size. Further, characters may be underlined.

FIG. 4(C) illustrates an example in which exaggeration display is performed, in addition to emphasis display. In the example illustrated in FIG. 4(C), characters are light up (oblique line portion), or a sound or vibration is made during the reception of a message. In this manner, the information conversion unit 150 may perform conversion into a presentation mode in which at least one of a sound, light, and vibration is generated when received information is displayed on the display unit 400.

Thereby, it is possible to strongly impress a receiver with the received information, rather than presenting received information by using only the sense of vision, and the like.

In addition, FIG. 4(D) illustrates an example in which the original text data is displayed by being converted into an euphemistic expression. The contents of the original information are displayed as they are by converting into a literary style into a polite expression, an estimation and conjecture expression, a passive expression, and the like. In this manner, the conversion into a soft literary style can alleviate a sense of intimidation, a sense of urgency, and the like that a receiver feels when acquiring the original information as it is.

FIG. 4(E) illustrates an example in which omission display is performed. In the example illustrated in FIG. 4(E), a receiver is notified of the reception of a message, for example, by displaying only a title and a summary of the original message. Further, when a portion “read the next” illustrated in FIG. 4(E) is clicked, an original message or a message converted into an euphemistic display mode as illustrated in FIG. 4(D) is displayed. In this manner, in the example illustrated in FIG. 4(E), the receiver can select whether or not to read the entire message or whether to read the message later.

Further, FIG. 4(F) illustrates an example in which display is performed by omitting the original message, as compared to the example illustrated in FIG. 4(E). In the example illustrated in FIG. 4(F), only an icon indicating a message and a title (or a summary) are displayed. Thereby, it is possible to impart only the reception of the message and minimum contents of the message to the receiver.

FIG. 4(G) illustrates an example in which information to be imparted to the receiver is minimized. In the example illustrated in FIG. 4(G), for example, only an icon indicating a message is displayed, or only nonverbal information such as vibration, a sound, or light is notified. As illustrated in FIG. 4(E) to FIG. 4(G), as the degree of omission of received information becomes stronger, a sense of urgency (urgency), a sense of intimidation, and the like to be given to the receiver decrease. This makes the receiver feel less unpleasant.

Next, a method of determining whether to perform conversion into any one display mode of the displays modes illustrated in FIG. 4(A) to FIG. 4(G) will be described. As described above, in this embodiment, a presentation mode of received information is converted on the basis of mental and physical information of a transmitter, mental and physical information of a receiver, contents of received information (content information), and at least one of positional information of the transmitter, and positional information of the receiver. First, a method of converting a presentation mode will be described with reference to the mental and physical information of the transmitter and the mental and physical information of the receiver.

The information conversion unit 150 determines which one of a positive state, a normal state, and a negative state a mental state is, on the basis of at least one of the mental and physical information of the receiver and the mental and physical information of the transmitter, and performs conversion of a presentation mode of the received information on the basis of the determined mental state.

Thereby, it is possible to perform the presentation of the received information in a presentation mode suitable for the respective mental states of the positive state, the normal state, and the negative state, and the like.

Specifically, in a case where the information conversion unit 150 determines that the mental state is a positive state, the information conversion unit performs conversion of the presentation mode of the received information so that the received information is displayed on the display unit 400, for example, in at least one display mode of the emphasis display as illustrated in FIG. 4(B) and the exaggeration display as illustrated in FIG. 4(C). In addition, in a case where the information conversion unit 150 determines that the mental state is a negative state, the information conversion unit performs conversion of the presentation mode of the received information so that the received information is displayed on the display unit 400, for example, in a display mode of the omission display as illustrated in FIG. 4(E) to FIG. 4(G).

Thereby, it is possible to perform the presentation of the received information by reflecting the transmitter's intention more strongly in a case where the mental state is a positive state, to perform the presentation of the received information so as not to hurt the receiver's feeling in a case where the mental state is a negative state, and the like.

Next, a method of converting a presentation mode on the basis of at least one of positional information of a transmitter and positional information of a receiver will be described. When the transmission and reception of information are performed, it is preferable to change a presentation mode of received information by also using the present positions (stay places) of the transmitter and the receiver. For example, a highly public place such as a hospital, a library, or a school is classified as a formal place, a home is classified as a personal place, and a place for work, a restaurant, and the like are classified as normal places. In the formal place, it is necessary to quietly perform the transmission and reception of information. On the other hand, in the personal place, the transmission and reception of information are permitted to be exaggeratedly performed to a certain extent. In the normal place, there is no particular problem even in a presentation mode on which the transmitter's intention is reflected as it is. In this manner, the presentation mode of the received information is changed depending on a place. Meanwhile, when it is determined whether the present positions of the transmitter and the receiver are formal places, normal places, or personal places, the place, out of the formal place, the normal place, and the personal place, which corresponds to each of the positions is determined in advance and is stored. The determination is performed by collating the stored data with the present positions of the transmitter and the receiver.

Thereby, it is possible to perform the presentation of the received information in a presentation mode suitable for at least one of the positions of the transmitter and the receiver, and the like.

At this time, the information conversion unit 150 determines which one of state of a formal state, a normal state, and a personal state the surrounding environment of the receiver is on the basis of the positional information, and performs conversion of the presentation mode of the received information on the basis of the determined surrounding environment.

Thereby, it is possible to perform the presentation of the received information in a presentation mode suitable for the surrounding environment of each of a formal state, a normal state, and a personal state, and the like. Meanwhile, the surrounding environment mainly means a place, but also includes information such as the size of a sound around the transmitter or the receiver, weather, temperature, humidity, how many people there are, and whether being indoor or outdoor.

Specifically, in a case where the information conversion unit 150 determines that the surrounding environment is in a normal state, the information conversion unit performs conversion of the presentation mode of the received information so that the received information is displayed on the display unit 400, for example, in at least one display mode of the emphasis display as illustrated in FIG. 4(B) and the exaggeration display as illustrated in FIG. 4(C). In addition, in a case where the information conversion unit 150 determines that the surrounding environment is in a personal state, the information conversion unit performs conversion of the presentation mode of the received information so that the received information is displayed on the display unit 400, for example, in a display mode of the omission display as illustrated in FIG. 4(E) to FIG. 4(G).

Thereby, it is possible to perform the presentation of the received information by reflecting the transmitter's intention more strongly in a case where the surrounding environment is in a normal state, to perform the presentation of the received information so as not to hurt the receiver's feeling in a case where the surrounding environment is in a personal state, and the like.

Next, a method of converting a presentation mode on the basis of contents of received information (content information) will be described. The contents of the received information can be classified into three types of formal, personal, and normal types. For example, in a case where the received information is text data, a document such as an invitation letter, a request form, an estimate form, or an invoice which includes a stamp or a signature, and the like are classified as formal contents, an insignificant exchange between colleagues, a chat, and the like are classified as personal contents, and a document that does not particularly include strict contents is classified as normal contents.

The information conversion unit 150 determines to which one of formal contents, normal contents, and personal contents the contents of the received information correspond, and performs conversion of the presentation mode of the received information on the basis of the determined contents.

Thereby, it is possible to perform the presentation of the received information in a presentation mode suitable for each of the formal contents, the normal contents, and the personal contents, and the like.

Specifically, in a case where the information conversion unit 150 determines that the contents of the received information are normal contents, the information conversion unit performs conversion of the presentation mode of the received information so that the received information is displayed on the display unit 400, for example, in at least one display mode of the emphasis display as illustrated in FIG. 4(B) and the exaggeration display as illustrated in FIG. 4(C). In addition, in a case where the information conversion unit 150 determines that the contents of the received information are personal contents, the information conversion unit performs conversion of the presentation mode of the received information so that the received information is displayed on the display unit 400, for example, in a display mode of the omission display as illustrated in FIG. 4(E) to FIG. 4(G).

Thereby, it is possible to perform the presentation of the received information by reflecting the transmitter's intention more strongly in a case where the contents of the received information are normal contents, to perform the presentation of the received information so as not to hurt the receiver's feeling in a case where the contents of the received information are personal contents, and the like.

Meanwhile, a method of distinguishing between formal contents, normal contents, and personal contents includes a method of designating contents by a transmitter himself or herself and a method of mechanically determining original information. In a case where the determination is mechanically performed, for example, a sentence included in the original information is taken out and is divided into words by morpheme analysis, and contents having a high proportion of polite forms and humble forms can be classified as formal contents. In contrast, contents including many informal expressions and katakana and hiragana characters can be classified as personal contents, and other contents can be classified as normal contents.

In the above description, a description has been given of an example in which a presentation mode of received information is converted by separately determining a mental state, a surrounding environment, and contents of information, but the conversion of the presentation mode is actually performed by complexly considering these pieces of information.

Specifically, the information conversion unit 150 determines a presentation mode of received information on the basis of a function F (S_{S}, P_{S}, S_{R}, P_{R}) using a parameter S_{S} obtained by digitizing a transmitter's mental state, a parameter P_{S} obtained by digitizing the transmitter's surrounding environment, a parameter S_{R} obtained by digitizing a receiver's mental state, and a parameter P_{R} obtained by digitizing the receiver's surrounding environment as arguments.

The function F is a function including a term obtained by linear combination of the parameter S_{S}, the parameter P_{S}, the parameter S_{R}, and the parameter P_{R}, and is a function, for example, as expressed by the following Expression (1).

F=β((α_{S}·S_{S}+β_{S}·β_{S})+(α_{R}·S_{R}+β_{R}·P_{R})+C) (1)

Meanwhile, in Expression (1), α_{S}, α_{R}, β_{S}, and β_{R} are any weighting coefficients, β is a normalization constant, and C is a parameter obtained by digitizing the state of contents. Regarding any weighting coefficients (α_{S}, α_{R}, β_{S}, β_{R}), a coefficient corresponding to a parameter having high priority is set to a large value, and a coefficient corresponding to a parameter having low priority is set to a small value. For example, when α_{S} is set to a large value, it is possible to increase the influence of the transmitter's mental state in determining a presentation mode of received information.

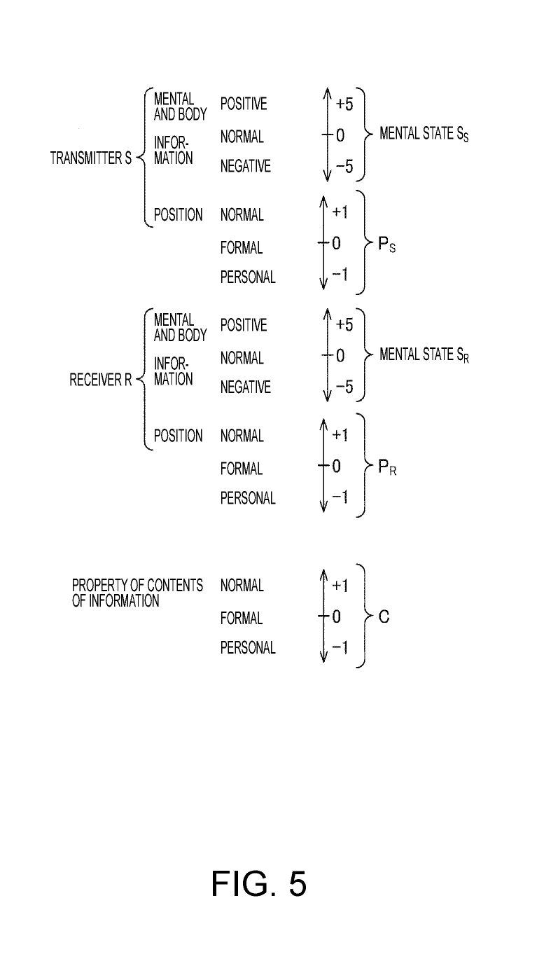

The parameters (S_{S}, P_{S}, S_{R}, P_{R}, C) are determined, for example, as illustrated in FIG. 5. The mental state S_{S} of the transmitter is set to +5 in a positive state, is set to 0 in a normal state, and is set to −5 in a negative state. The same is true of the mental state S_{R} of the receiver. In addition, the position P_{S} of the transmitter is set to +1 at a normal position, is set to 0 at a formal position, and is set to −1 at a personal position. The same is true of the position P_{R} of the receiver. The property C of contents of information is set to +1 in normal contents, is set to 0 in formal contents, and is set to −1 in personal contents. However, the values of the parameters are not limited thereto. In addition, a method of allocating a numerical value, a range in which the numerical value can be taken, and the like may be appropriately changed by the transmitter, the receiver, circumstances of the system, or the like.

In the above-described examples illustrated in FIG. 4(A) to FIG. 4(G), it is assumed that the value of F is set to be in a range between +5 and −5 by appropriately adjusting various variables and constants. An impartation method, that is, a presentation mode of received information is changed depending on this numerical value. A change to an impartation method (presentation mode) of preferentially imparting the fact of information being transmitted is roughly performed by putting off contents as the numerical value of F becomes smaller. In contrast, an impartation method of sufficiently imparting contents as the numerical value of F becomes larger and giving a strong impression having emphasis and exaggeration added thereto is adopted. Specifically, the presentation mode of FIG. 4(A) is determined when F=0, the presentation mode of FIG. 4(B) is determined when F=+1 to +3, the presentation mode of FIG. 4(C) is determined when F=+4 to +5, the presentation mode of FIG. 4(D) is determined when F=−1 to −2, the presentation mode of FIG. 4(E) is determined when F=−3, the presentation mode of FIG. 4(F) is determined when F=−4, and the presentation mode of FIG. 4(G) is determined when F=−5.

Thereby, it is possible to perform the conversion of a presentation mode by complexly considering the mental states and the surrounding environments of the transmitter and the receiver, and the like.

In addition, the function includes the term obtained by linear combination of the parameter S_{S}, the parameter P_{S}, the parameter S_{R}, and the parameter P_{R}, and thus it is possible to perform the determination of a presentation mode in consideration of the transmitter's mental state, the transmitter's surrounding environment, the receiver's mental state, and the receiver's surrounding environment, and the like.

More specifically, the function F is set to be the sum of parameters indicating the transmitter's conditions and parameters indicating the receiver's conditions for the following reason. In order for the receiver to consider the transmitter's intention, it is considered that information may be imparted according to the transmitter's intention as it is. However, since the receiver also has his or her feeling, it is necessary to perform a method of receiving information that also takes the receiver's intention into account. For example, when information is transmitted in a high motivation state (positive state) due to the transmitter being very excited, it is considered that the receiver may feel depressed and may not particularly desire to see or listen to the information (negative state). At this time, when a reception side receives information in the transmitter's high-tensed state (positive state) as it is, it is considered that the reception side does not feel very good. Consequently, the information is imparted with a slightly loose feeling in consideration of the receiver's depressed feeling. Thus, the information is not transmitted to the receiver with a flamboyant feeling, but some of the transmitter's excitement can be perceived through a title or the like. Accordingly, it is possible to realize the exchange of information which is capable of reflecting both the transmitter's intention and the receiver's intention. Similarly, considering another state, it is considered that it is possible to derive a best impartation method for both the transmitter and the receiver by taking the sum of states of both the transmitter and the receiver.

### 2.3. Modification Example

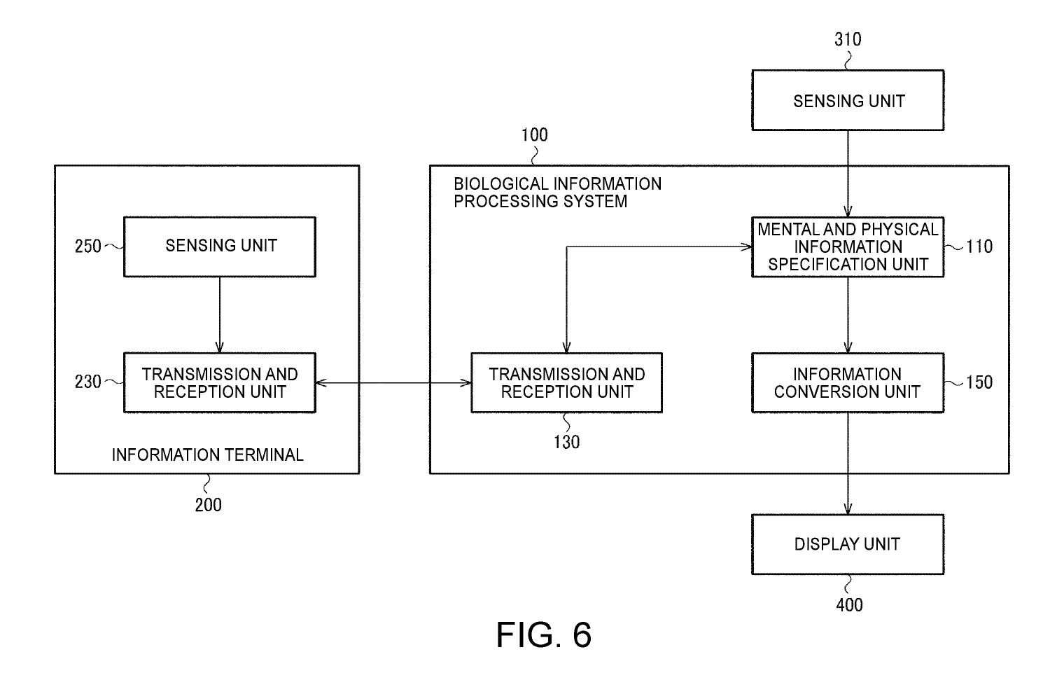

Next, FIG. 6 illustrates a modification example of this embodiment. In the above-described example, the information terminal 200 on a transmission side specifies mental and physical information of a transmitter and transmits the mental and physical information of the transmitter to the biological information processing system 100 on a reception side. On the other hand, in the modification example illustrated in FIG. 6, the information terminal 200 on the transmission side transmits biological information of the transmitter to the biological information processing system 100 on the reception side, and the biological information processing system 100 specifies the mental and physical information of the transmitter.

That is, the transmission and reception unit 130 receives the biological information of the transmitter. The mental and physical information specification unit 110 specifies the mental and physical information of the transmitter on the basis of the biological information of the transmitter, and specifies mental and physical information of a receiver on the basis of biological information of the receiver. The information conversion unit 150 performs a converting process of a presentation mode of received information, on the basis of contents of the received information received by the transmission and reception unit 130, the mental and physical information of the transmitter, and the mental and physical information of the receiver.

Thereby, it is possible to perform the converting process of the presentation mode of the received information, and the like even when the information terminal on the transmission side cannot specify the mental and physical information of the transmitter.

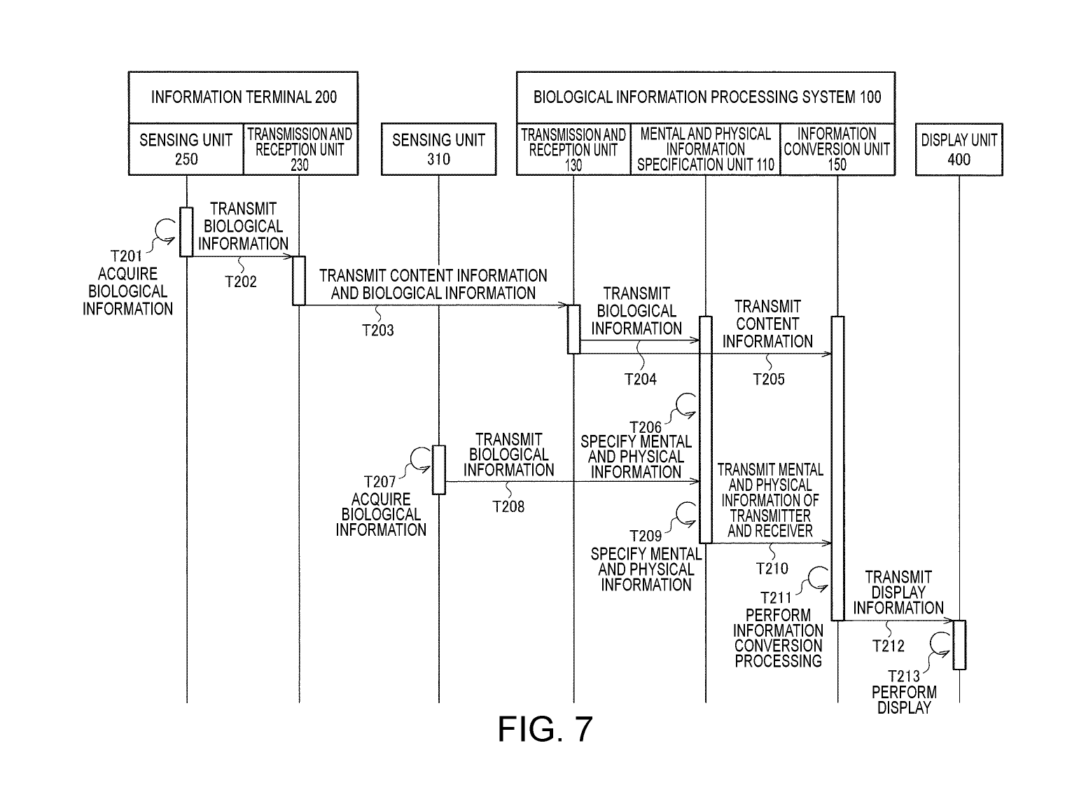

Next, a flow of processing according to this modification example will be described in detail with reference to a sequence diagram of FIG. 7.

First, the sensing unit 250 of the information terminal 200 acquires biological information of a transmitter of information (T201), and transmits the acquired biological information to the transmission and reception unit 230 (T202).

Next, the transmission and reception unit 230 of the information terminal 200 transmits content information to the transmission and reception unit 130 of the biological information processing system 100 together with the acquired biological information of the transmitter (T203). The transmission and reception unit 130 of the biological information processing system 100 which has received the content information and the biological information transmits the biological information to the mental and physical information specification unit 110 (T204), and transmits the content information to the information conversion unit 150 (T205). Further, the mental and physical information specification unit 110 specifies mental and physical information of the transmitter on the basis of the received biological information of the transmitter (T206).

Thereafter, the sensing unit 310 of the biological information processing system 100 acquires biological information of a receiver of information (T207), and transmits the acquired biological information to the mental and physical information specification unit 110 (T208). The mental and physical information specification unit 110 of the biological information processing system 100 specifies mental and physical information of the receiver on the basis of the biological information of the receiver (T209), and transmits the specified mental and physical information of the transmitter and the mental and physical information of the receiver to the information conversion unit 150 (T210).

Next, the information conversion unit 150 performs conversion of a presentation mode of received information on the basis of contents of the received information (content information), the mental and physical information of the transmitter, and the mental and physical information of the receiver (T211), and transmits the received information of which the presentation mode has been converted to the display unit 400 (T212). The display unit 400 displays the received information in the converted presentation mode (T213), and terminates the processing.

### 3. Second Embodiment

Next, a second embodiment will be described. In the first embodiment, the biological information processing system 100 on a reception side performs a converting process of a presentation mode of received information. However, in this embodiment, pieces of biological information of a transmitter and a receiver and content information (transmitted information) are transmitted to an external server system, and the server system performs a converting process of a presentation mode of received information and transmits the received information to a biological information processing system 100 in the converted presentation mode.

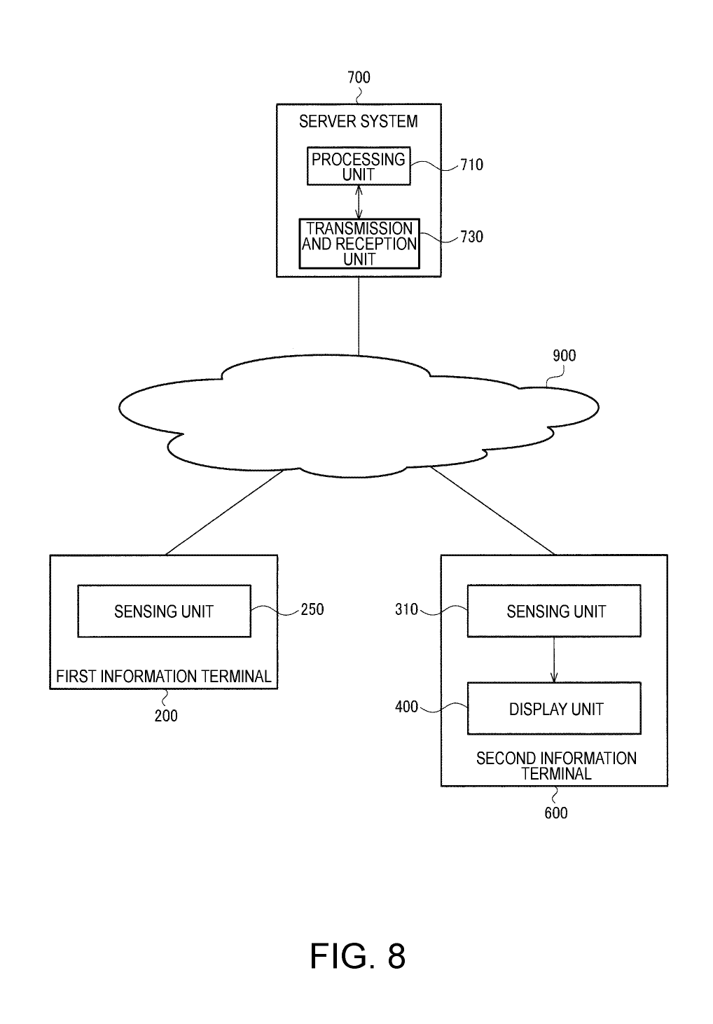

Here, a system configuration example according to this embodiment is illustrated in FIG. 8. A server system 700 according to this embodiment includes a transmission and reception unit 730 that performs communication between a first information terminal 200 and a second information terminal 600, and a processing unit 710. In addition, the first information terminal 200 includes at least a sensing unit 250. The second information terminal 600 includes at least a sensing unit 310 and a display unit 400. In this example, it is assumed that the first information terminal 200 transmits information to the second information terminal 600.

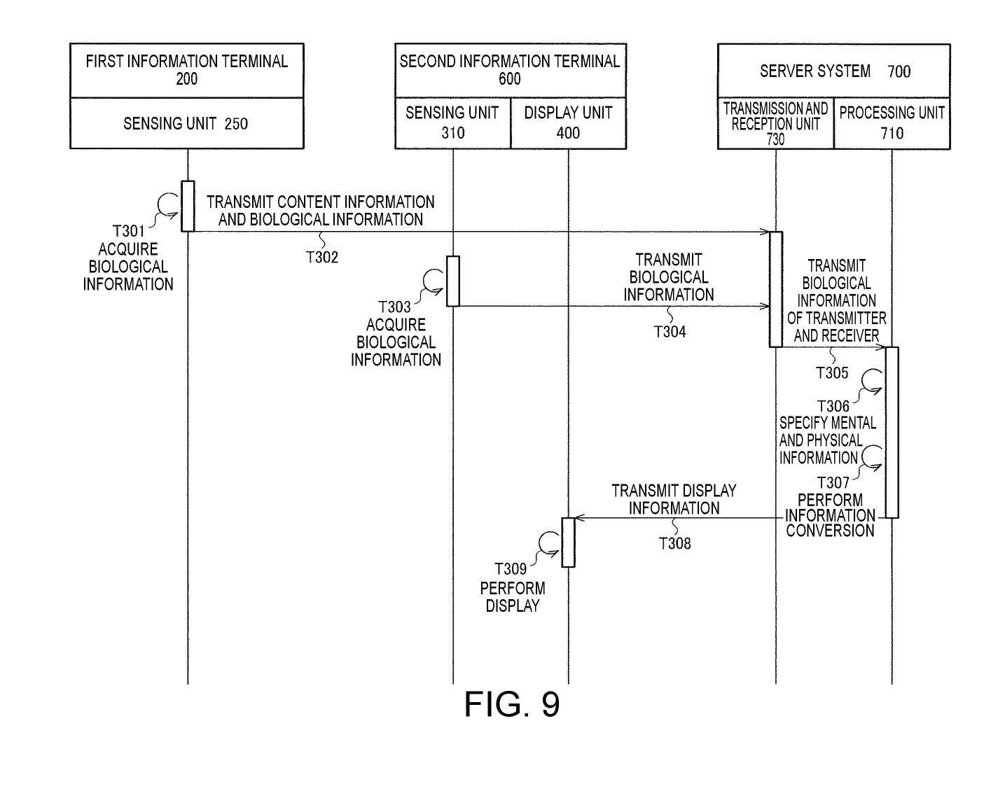

A flow of processing according to this embodiment is illustrated in a sequence diagram of FIG. 9. The transmission and reception unit 730 receives biological information of a transmitter which is measured by the sensing unit 250 of the first information terminal 200 and transmitted information from the first information terminal 200 (T301, T302). Similarly, the transmission and reception unit 730 receives biological information of a receiver which is measured by the sensing unit 310 of the second information terminal 600 from the second information terminal 600 (T303, T304).

The processing unit 710 specifies mental and physical information of the transmitter on the basis of the acquired biological information of the transmitter, and specifies mental and physical information of the receiver on the basis of the acquired biological information of the receiver (T305, T306). Thereafter, the processing unit 710 performs a converting process of a presentation mode of the transmitted information on the basis of contents of the transmitted information, the mental and physical information of the transmitter, and the mental and physical information of the receiver (T307).

Further, the processing unit 710 transmits the transmitted information (display information) of which the presentation mode has been converted to the display unit 400 of the second information terminal 600, and the display unit 400 displays display information (T309).

Thereby, it is possible to perform the conversion of the presentation mode of the transmitted information while reducing processing loads of the information terminals on the transmission and reception sides, and the like.

In addition, the server system 700 is constituted by a plurality of server devices such as a processing server and a data server. However, specific configurations of the server system 700, the first information terminal 200, and the second information terminal 600 are not limited thereto, and various modifications such as the omission of a portion of the components and the addition of other components can be made. In addition, some of the functions of the server system 700 may be dispersively realized by the first information terminal 200, the second information terminal 600, and the like.

In the example of FIG. 8, the first information terminal 200, the second information terminal 600, and the server system 700 are communicatively connected to each other through a network 900 including at least one of a wireless and wired networks. The network 900 may include various networks having different communication standards. For example, the first information terminal 200 and the second information terminal 600 are connected to each other by Bluetooth (registered trademark), a wireless LAN, or the like, and connection between the first information terminal 200 and the server system 700 and connection between the second information terminal 600 and the server system 700 are performed by the Internet. However, this embodiment is not limited thereto.

### 4. Process of Estimating Mental State

Next, a process of estimating mental state information will be described in detail. In particular, a description will be given of a specific method of estimating the state of introspective mind, such as the level of motivation or user evaluation for an action, among pieces of mental state information from a psychological state, such as a tensed state, a relaxed state, or an awakening state, and the like. Meanwhile, the user evaluation for an action means information indicating, for example, the degree at which a user feels happy to perform a certain action or regrets to perform the action when the user performs the action. The user evaluation may be the user's conscious feeling or may be the user's unconscious feeling.

First, a psychological state such as a tensed state, a relaxed state, or an awakening state is estimated from biological information such as heartbeats (pulses), blood pressure, and body temperature. Here, a method of estimating the psychological state from heartbeats (pulses) will be described. The heartbeats (pulsation of the heart) indicate an activity state of the heart. The heart is influenced by the sympathetic nerve, and thus it is possible to estimate the activity state of the sympathetic nerve from the state of the heartbeat.

Frequency analysis is generally used as a method of analyzing the influence of the sympathetic nerve and the parasympathetic nerve in the heartbeat. Frequency analysis (FFT) of temporal changes in a distance between peaks (R-R interval) of a graph regarding change in heartbeat is performed to perform division into a low frequency component LF and a high frequency component HF, thereby performing analysis.

It is possible to quantify the influence of the sympathetic nerve and the parasympathetic nerve from the low frequency component LF and the high frequency component HF. The quantified value corresponds to the degree of psychological load stress, and thus it is possible to estimate a psychological state such as being relaxed, being excited, or feeling good from the size of a numerical value.

Incidentally, the quantified psychological state such as a tensed state does not necessarily conform to a mental state such as being moved (high motivation state) or feeling good (user evaluation) when watching TV. Even when the numerical value indicates an excited state or a relaxed state, it is not possible to determine that the state is necessarily a comfortable state with only one psychological index in many cases. For this reason, it is considered that it is necessary to perform comprehensive determination from a user's conditions at that time by also considering other psychological states.

Next, a method of estimating a mental state, such as the level of motivation or user evaluation for an action result, from a plurality of quantified psychological states will be described.

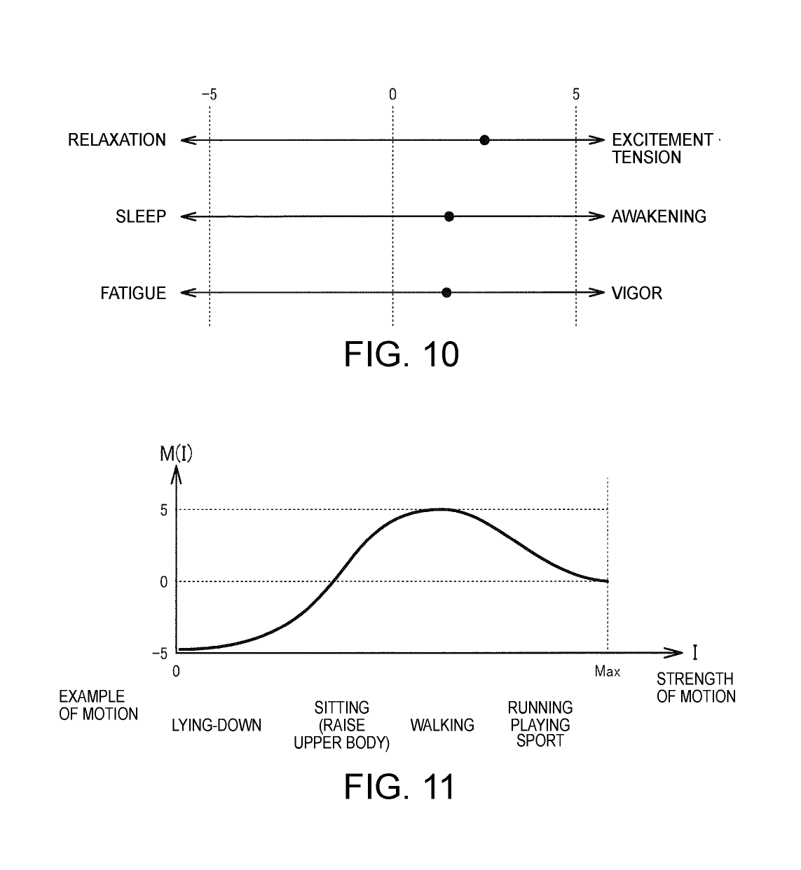

First, a plurality of psychological states are converted into numerical values through frequency analysis of a heart rate, or the like from a heart rate (HR) which is one of pieces of biological information. Here, a description will be given of an example in which three indexes of A: relaxation-excitement and tension, B: sleep-awakening, and C: fatigue-vigor (degree of physical strength) illustrated in FIG. 10 are used. Specifically, A uses a ratio of HF to LF, B uses HR (base HR) in a rest state, and C uses the state of a maximum value of HR (body is tired when the latest maximum value is small, and thus feeling tired is also inferred), and the like.

The indexes of A, B, and C indicate opposite states. Accordingly, for example, in a case of A, a maximum value in a relaxed state and a maximum value in an excited state are respectively set to numerical values of −5 and 5 by setting an intermediate point between relaxation and excitement to 0. The same is true of B and C.

Next, not only the plurality of psychological states but also a motion state of the person is used for the estimation of a mental state of the person. Even in the same psychological state, as the intensity of a motion increases, the influence on a heartbeat in the psychological state decreases. For this reason, the influence of the intensity of a motion is expressed as a function M(I) illustrated in FIG. 11, and is used for the estimation of a mental state. The horizontal axis in FIG. 11 represents a motion intensity I (Mets and the like), and the vertical axis represents a motion state M(I) which is a function of I. In a state where a body rarely moves such as a lying-down state, a seating state, or a state of an upper body being raised, M(I) becomes smaller than in another motion state. M(I) is maximized in a walking state, and M(I) becomes smaller when the intensity of a motion is obtained, such as in a running state or a state of playing sport, than in the walking state. Meanwhile, in the example illustrated in FIG. 11, M(I) ranges from −5 to 5 in conformity to the example illustrated in FIG. 10.

A mental state X is quantified from the psychological state and the motion state that are obtained in this manner, by the following expression. Meanwhile, α, β, and γ are variables, and can be appropriately adjusted. The same signs are used, but are parameters different from the parameters of Expression (1).

X=(αA+βB+γC+ . . . )+M(I) (2)

In the example illustrated in FIG. 10, the relations of A=3 and B=C=2 are established. Here, α, β, and γ are all set to 1, and a user's sitting state is set. Since M(I)=0 in the sitting state, the relation of X=7 is established. That is, the level of motivation can be specified as 7.

When the motivation is high, the user is excited in many cases. However, there is a possibility that the user is in an excited state simply due to some stimulus when the user is half asleep or is not vigorous. In this case, it cannot be said that the user is sufficiently motivated. In addition, when the user is in motion, the pulse becomes fast by the motion itself. Accordingly, it is considered that the influence of a willing feeling is reduced, and thus it is necessary to take off the influence of the motion. In this manner, it is possible to estimate a more accurate amount of motivation in consideration of the plurality of psychological states and the motion.

As described above, it is possible to quantify a mental state from a psychological state based on heartbeats and a motion state based on movement. Similarly, regarding cases of blood pressure and body temperature other than heartbeats, A, B, and C are obtained by estimation based on other pieces of biological information. In a case of a plurality of pieces of biological information, for example, A, B, and C are obtained from heartbeats, A′, B′, and C′ are obtained from blood pressure, and a mental state is obtained by linear combination thereof.

As described above, the mental and physical information specification unit 110 (mental and physical information specification unit 210) according to this embodiment estimates the level of motivation of a user as mental state information. The level of motivation may be directly estimated from biological information, or may be estimated from a tensed state and the like.

### 5. Configuration Example of Information Terminal

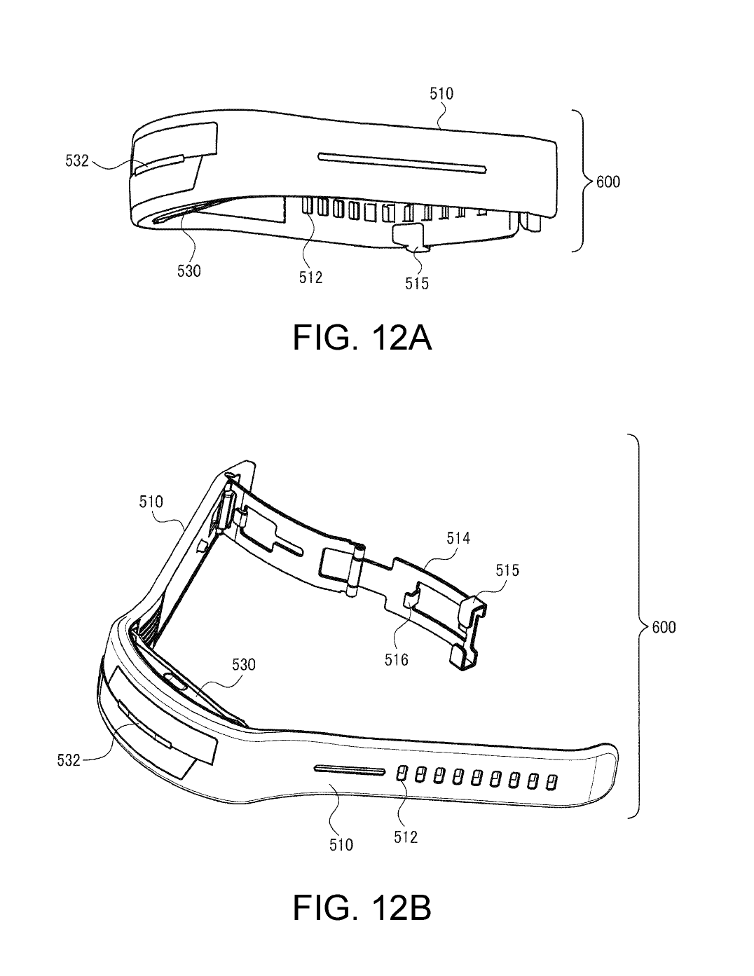

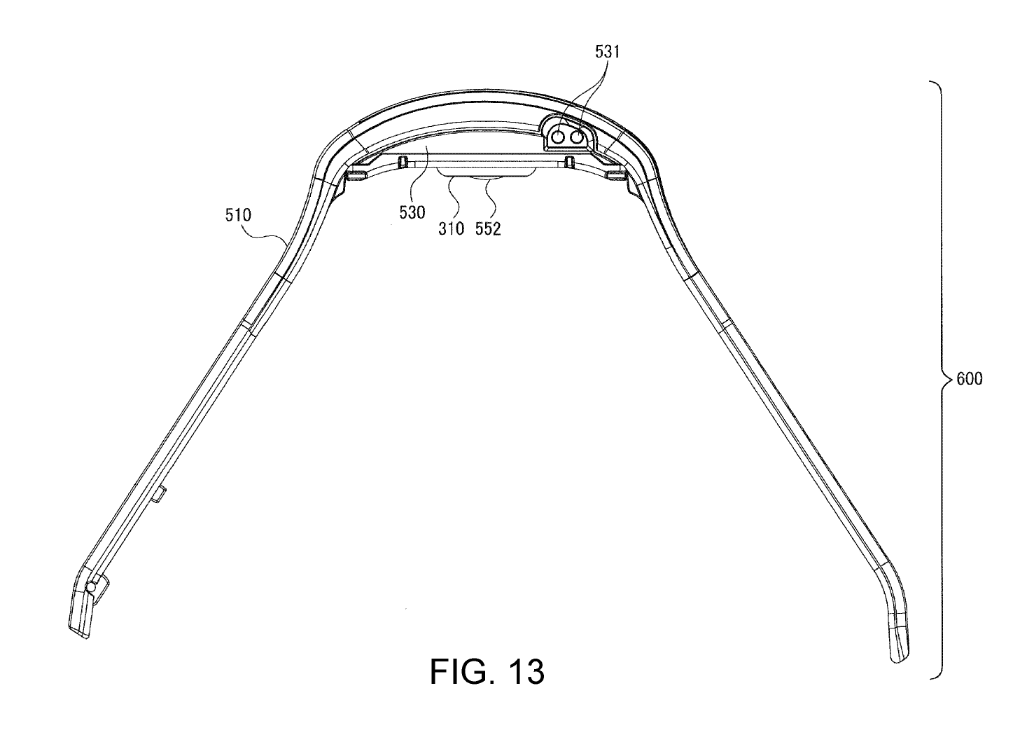

FIG. 12(A), FIG. 12(B), and FIG. 13 illustrate the exterior of an information terminal (first information terminal 200 or second information terminal 600) according to this embodiment. FIG. 12(A) is a diagram when the information terminal is seen from the front side, FIG. 12(B) is a diagram when the information terminal is seen from the upper side, and FIG. 13 is a diagram when the information terminal is seen from the side.

As illustrated in FIG. 12(A) to FIG. 13, the information terminal according to this embodiment includes a band portion 510, a case portion 530, and a sensing unit 310. The case portion 530 is attached to the band portion 510. The sensing unit 310 is provided in the case portion 530. Meanwhile, the information terminal according to this embodiment is not limited to having a configuration illustrated in FIG. 12(A) to FIG. 13 and the like, and various modifications such as the omission of a portion of the components, replacement with other components, or the addition of other components can be made.

The band portion 510 is a portion wound around a user's wrist to wear the information terminal. The band portion 510 includes holes 512 and a buckle portion 514. The buckle portion 514 includes a band insertion portion 515 and a projection portion 516. The user inserts one end of the band portion 510 into the band insertion portion 515 of the buckle portion 514 and inserts the projection portion 516 of the buckle portion 514 into the hole 512 of the band portion 510, and thus the user can wear the information terminal on his or her wrist. In this case, the magnitude of pressing on the sensing unit 310 (pressing on the surface of the wrist) is adjusted depending on into which hole 512 the projection portion 516 is inserted.

The case portion 530 is equivalent to the main body of the information terminal. Various components such as the sensing unit 310 and the biological information processing system 100 (including the mental and physical information specification unit 110, the transmission and reception unit 130, the information conversion unit 150, and the like) are provided inside the case portion 530. That is, the case portion 530 is a housing that accommodates these components.

The case portion 530 is provided with a light emitting window portion 532. The light emitting window portion 532 is constituted by a light transmission member. The case portion 530 is provided with a light emitting portion (LED) mounted on a flexible substrate, and light emitted from the light emitting portion is emitted to the outside of the case portion 530 through the light emitting window portion 532.

As illustrated in FIG. 13, the case portion 530 is provided with a terminal portion 531. When the information terminal is mounted on a cradle not shown in the drawing, a terminal portion of the cradle and the terminal portion 531 of the case portion 530 are electrically connected to each other. Thereby, it is possible to charge a secondary battery (battery) provided in the case portion 530.

Meanwhile, a portion or the majority of the processing in the biological information processing system, the server system, and the like according to this embodiment may be implemented by a program. In this case, the biological information processing system, the server system, and the like of the embodiment is implemented by a processor such as a CPU executing the program. Specifically, the program stored in a non-transitory information storage device is read out, and the processor such as a CPU executes the read-out program. Here, the information storage device (computer-readable device) stores a program, data, and the like, and the functions thereof can be implemented by an optical disc (a DVD, a CD, or the like), an HDD (hard disk drive) or a memory (a card-type memory, a ROM, or the like), or the like. The processor such as a CPU performs various processes according to this embodiment on the basis of the program (data) stored in the information storage device. That is, in the information storage device, a program for causing a computer (a device including an operation unit, a processing unit, a storage unit, and an output unit) to function as each unit of this embodiment (program for causing a computer to execute processing of each unit) is stored.

In addition, the biological information processing system, the server system, and the like according to this embodiment may include a processor and a memory. Here, the processor may be, for example, a central processing unit (CPU). However, the processor is not limited to the CPU, and any of various other processors such as a graphics processing unit (GPU) and a digital signal processor (DSP) can be used. In addition, the processor may be a hardware circuit based on an application specific integrated circuit (ASIC). In addition, the memory stores a computer-readable command, and each of the units of the biological information processing system, the server system, and the like according to this embodiment is implemented by the command being executed by the processor. Here, the memory may be a semiconductor memory such as a static random access memory (SRAM) or a dynamic random access memory (DRAM), or may be a register, a hard disk, or the like. Here, the command may be a command of a command set constituting a program or may be a command for giving an instruction to the hardware circuit of the processor.

While this embodiment has been described in detail, one skilled in the art can easily understand that a number of modifications can be made without substantially departing from the new matters and effects of the invention. Therefore, all such modification examples are included in the scope of the invention. For example, a term described at least once along with a different term having a broader meaning or the same meaning in the description or drawings can be replaced with the different term at any location in the description or drawings. In addition, the configurations and operations of the biological information processing system and the server system are not limited to those described in this embodiment and can be modified in various ways.

### REFERENCE SIGNS LIST

100: biological information processing system
110: mental and physical information specification unit
130: transmission and reception unit
150: information conversion unit
200: information terminal
210: mental and physical information specification unit
230: transmission and reception unit
250: sensing unit
310: sensing unit
400: display unit
510: band portion
512: hole
514: buckle portion
515: band insertion portion
516: projection portion
530: case portion
531: terminal portion
532: light emitting window portion
600: information terminal
700: server system
710: processing unit
730: transmission and reception unit
900: network

1. A biological information processing system comprising:
a mental and physical information specification unit that specifies mental and physical information on the basis of biological information measured by a sensing unit;
a transmission and reception unit that performs transmission and reception of information; and
an information conversion unit that performs a converting process of a presentation mode of received information, on the basis of contents of the received information received by the transmission and reception unit, mental and physical information of a transmitter, and mental and physical information of a receiver.

2. The biological information processing system according to claim 1,
wherein the information conversion unit performs conversion of the presentation mode of the received information on the basis of the contents of the received information, the mental and physical information of the transmitter, the mental and physical information of the receiver, and at least one of positional information of the transmitter and positional information of the receiver.

3. The biological information processing system according to claim 1,
wherein the information conversion unit performs conversion of the presentation mode of the received information so that the received information is displayed on a display unit in at least one display mode of emphasis display, exaggeration display, and omission display.

4. The biological information processing system according to claim 1,
wherein the information conversion unit determines which one of a positive state, a normal state, and a negative state a mental state is, on the basis of at least one of the mental and physical information of the receiver and the mental and physical information of the transmitter, and performs conversion of the presentation mode of the received information on the basis of the determined mental state.

5. The biological information processing system according to claim 4,
wherein the information conversion unit performs conversion of the presentation mode of the received information so that the received information is displayed on the display unit in at least one display mode of the emphasis display and the exaggeration display in a case where it is determined that the mental state is the positive state, and performs conversion of the presentation mode of the received information so that the received information is displayed on the display unit in the display mode of the omission display in a case where it is determined that the mental state is the negative state.

6. The biological information processing system according to claim 2,
wherein the information conversion unit determines which one of state of a formal state, a normal state, and a personal state a surrounding environment of the receiver is, on the basis of the positional information, and performs conversion of the presentation mode of the received information on the basis of the determined surrounding environment.

7. The biological information processing system according to claim 6,
wherein the information conversion unit performs conversion of the presentation mode of the received information so that the received information is displayed on a display unit in at least one display mode of emphasis display and exaggeration display in a case where it is determined that the surrounding environment is in the normal state, and performs conversion of the presentation mode of the received information so that the received information is displayed on the display unit in the display mode of the omission display in a case where it is determined that the surrounding environment is in the personal state.

8. The biological information processing system according to claim 1,
wherein the information conversion unit determines which one of formal contents, normal contents, and personal contents the contents of the received information are, and performs conversion of the presentation mode of the received information on the basis of the determined contents.

9. The biological information processing system according to claim 8,
wherein the information conversion unit performs conversion of the presentation mode of the received information so that the received information is displayed on the display unit in at least one display mode of the emphasis display and the exaggeration display in a case where it is determined that the contents of the received information are the normal contents, and performs conversion of the presentation mode of the received information so that the received information is displayed on the display unit in the display mode of the omission display in a case where it is determined that the contents of the received information are the personal contents.

10. The biological information processing system according to claim 1,
wherein the information conversion unit performs conversion into the presentation mode in which at least one of a sound, light, and vibration is generated when the received information is displayed on the display unit.

11. The biological information processing system according to claim 1,
wherein the transmission and reception unit receives the mental and physical information of the transmitter, and the information conversion unit performs the converting process of the presentation mode of the received information on the basis of the contents of the received information, the mental and physical information of the transmitter, and the mental and physical information of the receiver which are received by the transmission and reception unit.

12. The biological information processing system according to claim 1,
wherein the transmission and reception unit receives the biological information of the transmitter, the mental and physical information specification unit specifies the mental and physical information of the transmitter on the basis of the biological information of the transmitter and specifies the mental and physical information of the receiver on the basis of the biological information of the receiver, and the information conversion unit performs the converting process of the presentation mode of the received information on the basis of the contents of the received information, the mental and physical information of the transmitter, and the mental and physical information of the receiver which are received by the transmission and reception unit.

13. The biological information processing system according to claim 1,
wherein the information conversion unit determines the presentation mode of the received information on the basis of a function F (S_{S}, P_{S}, S_{R}, P_{R}) using a parameter S_{S} obtained by digitizing the transmitter's mental state, a parameter P_{S} obtained by digitizing the transmitter's surrounding environment, a parameter S_{R} obtained by digitizing the receiver's mental state, and a parameter P_{R} obtained by digitizing the receiver's surrounding environment as arguments.

14. The biological information processing system according to claim 13,
wherein the function F is a function including a term obtained by linear combination of the parameter S_{S}, the parameter P_{S}, the parameter S_{R}, and the parameter P_{R}.

15. A server system comprising:
a transmission and reception unit that performs communication between a first information terminal and a second information terminal; and
a processing unit,
wherein the transmission and reception unit receives biological information of a transmitter which is measured by a sensing unit of the first information terminal and transmitted information from the first information terminal, and receives biological information of a receiver measured by a sensing unit of the second information terminal from the second information terminal, and
wherein the processing unit specifies mental and physical information of the transmitter on the basis of the acquired biological information of the transmitter, specifies mental and physical information of the receiver on the basis of the acquired biological information of the receiver, and performs a converting process of a presentation mode of the transmitted information on the basis of contents of the transmitted information, the mental and physical information of the transmitter, and the mental and physical information of the receiver.

16. An information processing method comprising:
specifying mental and physical information on the basis of biological information measured by a sensing unit;
performing transmission and reception of information; and
performing a converting process of a presentation mode of received information on the basis of contents of the received information, mental and physical information of a transmitter, and mental and physical information of a receiver.
